(19) United States
(12) Patent Application Publication
(10) Pub. No.: US 2012/0187383 A1
(43) Pub. Date: Jul. 26, 2012

(54) ELECTROACTIVE COMPOUND AND COMPOSITION AND ELECTRONIC DEVICE MADE WITH THE COMPOSITION

(75) Inventors: WEIYING GAO, Landenberg, PA (US); Weishi Wu, Landenberg, PA (US); Kalindi Dogra, Wilmington, DE (US); Vsevolod Rostovtsev, Swarthmore, PA (US); Kerwin D. Dobbs, Wilmington, DE (US)

(73) Assignee: E.I. DU PONT DE NEMOURS AND COMPANY, Wilmington, DE (US)

(21) Appl. No.: 13/195,044

(22) Filed: Aug. 1, 2011

Related U.S. Application Data
(60) Provisional application No. 61/372,488, filed on Aug. 11, 2010.

(51) Int. Cl. H01L 51/54; C09K 11/06; C07C 211/59; C07C 15/24; C07C 255/51

(52) U.S. Cl. 257/40; 585/26; 558/419; 564/426; 252/301.16; 257/E51.026

(57) ABSTRACT

There is provided an a host material and a dopant material, wherein the host material is a compound having one of Formulae I-VI:

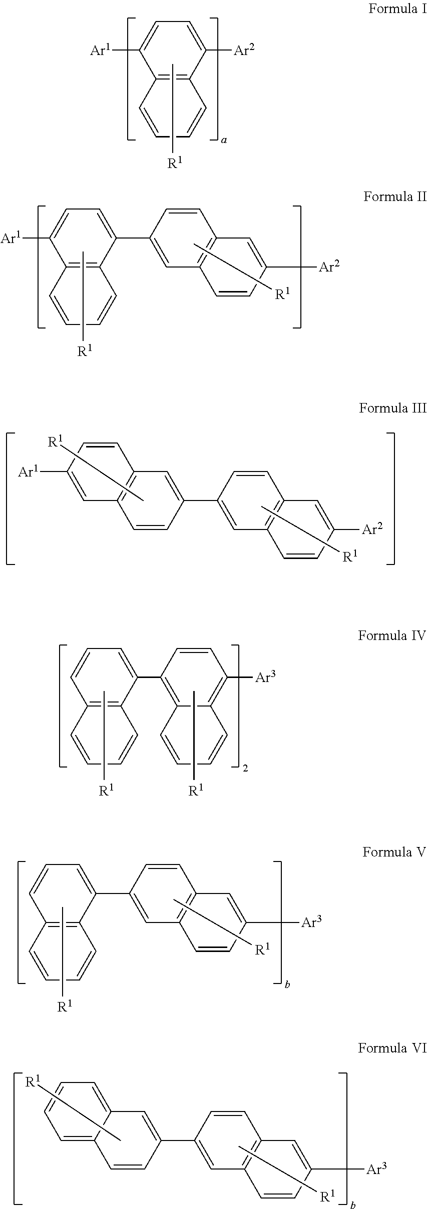

In the formulae, R¹ is the same or different at each occurrence and represents an optional substituent which may be present at any or all of the available sites and may be D, alkyl, aryl, alkoxy, aryloxy, oxyalkyl, alkenyl, silyl, or siloxane; Ar¹, Ar², and Ar³ are the same or different at each occurrence and are aryl groups; a is an integer from 2-6; and b is an integer from 1-3.

### RELATED APPLICATION DATA

This application claims priority under 35 U.S.C. §119(e) from U.S. Provisional Application No. 61/372,488 filed on Aug. 11, 2010, which is incorporated by reference herein in its entirety.

### BACKGROUND INFORMATION

1. Field of the Disclosure

This disclosure relates in general to electroactive compositions that are useful in organic electronic devices.

2. Description of the Related Art

In organic electroactive electronic devices, such as organic light emitting diodes (“OLED”), that make up OLED displays, the organic active layer is sandwiched between two electrical contact layers in an OLED display. In an OLED, the organic electroactive layer emits light through the light-transmitting electrical contact layer upon application of a voltage across the electrical contact layers.

It is well known to use organic electroluminescent compounds as the active component in light-emitting diodes. Simple organic molecules, conjugated polymers, and organometallic complexes have been used.

Devices that use electroactive materials frequently include one or more charge transport layers, which are positioned between an electroactive (e.g., light-emitting) layer and a contact layer (hole-injecting contact layer). A device can contain two or more contact layers. A hole transport layer can be positioned between the electroactive layer and the hole-injecting contact layer. The hole-injecting contact layer may also be called the anode. An electron transport layer can be positioned between the electroactive layer and the electron-injecting contact layer. The electron-injecting contact layer may also be called the cathode. Charge transport materials can also be used as hosts in combination with the electroactive materials.

There is a continuing need for new materials and compositions for electronic devices.

### SUMMARY

There is provided an electroactive compound having one of Formulae I through VI

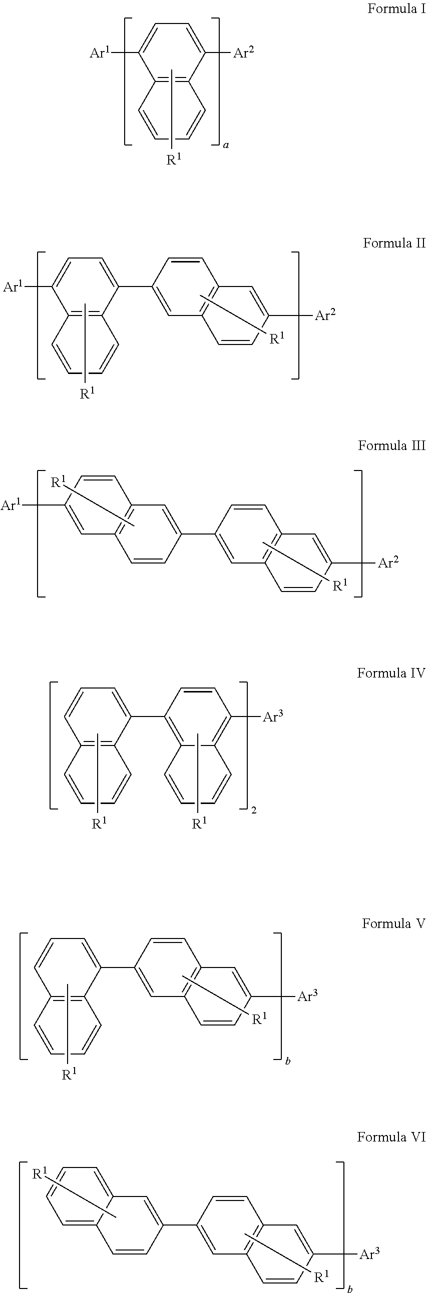

wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

There is further provided an electroactive composition comprising a host material and an electroluminescent dopant material, wherein the host material is a compound having one of Formulae I-VI, shown above.

There is also provided an organic electronic device comprising two electrical contact layers with an organic electroactive layer therebetween, wherein the electroactive layer comprises the electroactive composition described above.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 includes an illustration of an exemplary organic device.

FIG. 2 includes an illustration of an exemplary organic device.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description first addresses Definitions and Clarification of Terms followed by the Electroactive Compound, the Electroactive Composition, the Electronic Device, and finally Examples.

### 1. DEFINITIONS AND CLARIFICATION OF TERMS

Before addressing details of embodiments described below, some terms are defined or clarified.

The term “alkyl” is intended to mean a group derived from an aliphatic hydrocarbon. In some embodiments, the alkyl group has from 1-20 carbon atoms.

The term “aryl” is intended to mean a group derived from an aromatic hydrocarbon. The term “aromatic compound” is intended to mean an organic compound comprising at least one unsaturated cyclic group having delocalized pi electrons. The term is intended to encompass both aromatic compounds having only carbon and hydrogen atoms, and heteroaromatic compounds wherein one or more of the carbon atoms within the cyclic group has been replaced by another atom, such as nitrogen, oxygen, sulfur, or the like. In some embodiments, the aryl group has from 4-30 carbon atoms.

The term “charge transport,” when referring to a layer, material, member, or structure is intended to mean such layer, material, member, or structure facilitates migration of such charge through the thickness of such layer, material, member, or structure with relative efficiency and small loss of charge. Hole transport materials facilitate positive charge; electron transport materials facilitate negative charge. Although light-emitting materials may also have some charge transport properties, the term “charge transport layer, material, member, or structure” is not intended to include a layer, material, member, or structure whose primary function is light emission.

The term “deuterated” is intended to mean that at least one H has been replaced by D. The term “deuterated analog” refers to a structural analog of a compound or group in which one or more available hydrogens have been replaced with deuterium. In a deuterated compound or deuterated analog, the deuterium is present in at least 100 times the natural abundance level.

The term “dopant” is intended to mean a material, within a layer including a host material, that changes the electronic characteristic(s) or the targeted wavelength(s) of radiation emission, reception, or filtering of the layer compared to the electronic characteristic(s) or the wavelength(s) of radiation emission, reception, or filtering of the layer in the absence of such material.

The term “electroactive” as it refers to a layer or a material, is intended to indicate a layer or material which electronically facilitates the operation of the device. Examples of electroactive materials include, but are not limited to, materials which conduct, inject, transport, or block a charge, where the charge can be either an electron or a hole, or materials which emit radiation or exhibit a change in concentration of electron-hole pairs when receiving radiation. Examples of inactive materials include, but are not limited to, planarization materials, insulating materials, and environmental barrier materials.

The term “electroluminescence” refers to the emission of light from a material in response to an electric current passed through it. “Electroluminescent” refers to a material that is capable of electroluminescence.

The term “emission maximum” is intended to mean the highest intensity of radiation emitted. The emission maximum has a corresponding wavelength.

The term “fused aryl” refers to an aryl group having two or more fused aromatic rings.

The prefix “hetero” indicates that one or more carbon atoms has been replaced with a different atom. In some embodiments, the heteroatom is O, N, S, or combinations thereof.

The term “host material” is intended to mean a material, usually in the form of a layer, to which a dopant may or may not be added. The host material may or may not have electronic characteristic(s) or the ability to emit, receive, or filter radiation.

The term “layer” is used interchangeably with the term “film” and refers to a coating covering a desired area. The term is not limited by size. The area can be as large as an entire device or as small as a specific functional area such as the actual visual display, or as small as a single sub-pixel. Layers and films can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, and continuous nozzle coating. Discontinuous deposition techniques include, but are not limited to, ink jet printing, gravure printing, and screen printing.

The term “organic electronic device,” or sometimes just “electronic device,” is intended to mean a device including one or more organic semiconductor layers or materials.

The term “photoactive” refers to a material that emits light when activated by an applied voltage (such as in a light emitting diode or chemical cell) or responds to radiant energy and generates a signal with or without an applied bias voltage (such as in a photodetector).

The term “siloxane” refers to the group (RO)₃Si—, where R is H, D, C1-20 alkyl, or fluoroalkyl.

The term “silyl” refers to the group —SiR₃, where R is the same or different at each occurrence and is an alkyl group or an aryl group.

The prefix “hetero” indicates that one or more carbon atoms have been replaced with a different atom. In some embodiments, the different atom is N, O, or S. The prefix “fluoro” indicates that one or more hydrogen atoms have been replaced with a fluorine atom.

Unless otherwise indicated, all groups can be unsubstituted or substituted. Unless otherwise indicated, all groups can be linear, branched or cyclic, where possible. In some embodiments, the substituents are D, alkyl, alkoxy, aryl, silyl, or siloxane.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the disclosed subject matter hereof, is described as consisting essentially of certain features or elements, in which embodiment features or elements that would materially alter the principle of operation or the distinguishing characteristics of the embodiment are not present therein. A further alternative embodiment of the described subject matter hereof is described as consisting of certain features or elements, in which embodiment, or in insubstantial variations thereof, only the features or elements specifically stated or described are present. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Group numbers corresponding to columns within the Periodic Table of the elements use the “New Notation” convention as seen in the CRC Handbook of Chemistry and Physics, 81^{st} Edition (2000-2001).

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

To the extent not described herein, many details regarding specific materials, processing acts, and circuits are conventional and may be found in textbooks and other sources within the organic light-emitting diode display, photodetector, photovoltaic, and semiconductive member arts.

### 2. ELECTROACTIVE COMPOUND

The electroactive compound has one of Formulae I through VI

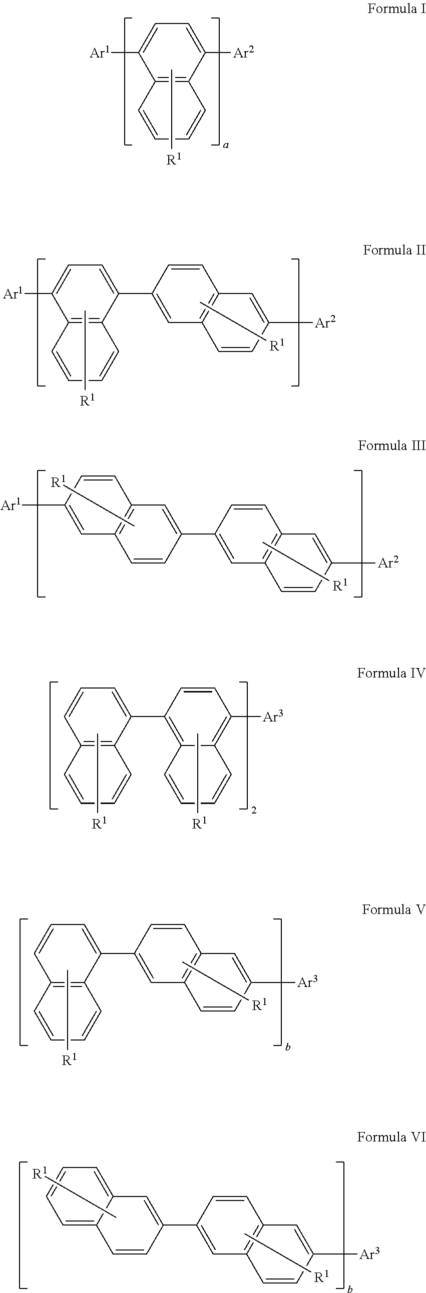

wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

In some embodiments of Formulae I-VI, aryl groups Ar¹, Ar², and Ar³ and any aryl substituents have no more than two fused rings. In some embodiments, aryl groups have one or more rings that are phenyl or naphthyl. The aryl groups may be unsubstituted or substituted. In some embodiments, the substituted aryl group has one or more substituents that are D, alkyl, alkoxy, phenyl, naphthyl, silyl, siloxane, or combinations thereof.

In some embodiments of Formulae I-III, Ar¹ and Ar² are the same or different and have Formula a:

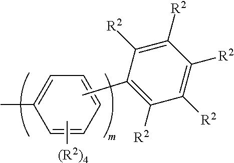

where:
R² is the same or different at each occurrence and is H, D, alkyl, alkoxy, siloxane or silyl, or adjacent R² groups may be joined together to form an aromatic ring; and
m is the same or different at each occurrence and is an integer from 1 to 6.

In some embodiments, Ar¹ and Ar² are the same or different and are phenyl, biphenyl, naphthylphenyl, naphthylbiphenyl, terphenyl, or quaterphenyl. The terphenyl and quaterphenyl groups can be bonded together in a linear arrangement (para bonding) or a non-linear arrangement.

In some embodiments of Formulae IV-VI, Ar³ has Formula b:

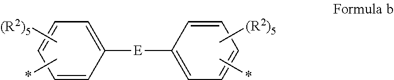

where:
R² is the same or different at each occurrence and is H, D, alkyl, alkoxy, siloxane or silyl, or adjacent R² groups may be joined together to form an aromatic ring;
E is a single bond, C(R³)₂, O, Si(R³)₂, or Ge(R³)₂; and
R³ is alkyl or aryl, or two R³ groups can join together to form a non-aromatic ring.

In some embodiments of Formulae I-VI, there is at least one substituent on at least one aryl ring. In some embodiments, the substituent is D, alkyl, alkoxy, siloxane or silyl.

In some embodiments, the electroactive compound having one of Formula I-VI is deuterated. In some embodiments, the electroactive compound is at least 10% deuterated. By “% deuterated” or “% deuteration” is meant the ratio of deuterons to the total of hydrogens plus deuterons, expressed as a percentage. The deuteriums may be on the same or different aryl groups. In some embodiments, the electroactive compound is at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

Some examples of the electroactive compound described herein include, but are not limited to, Compound A1 through A17, shown below.

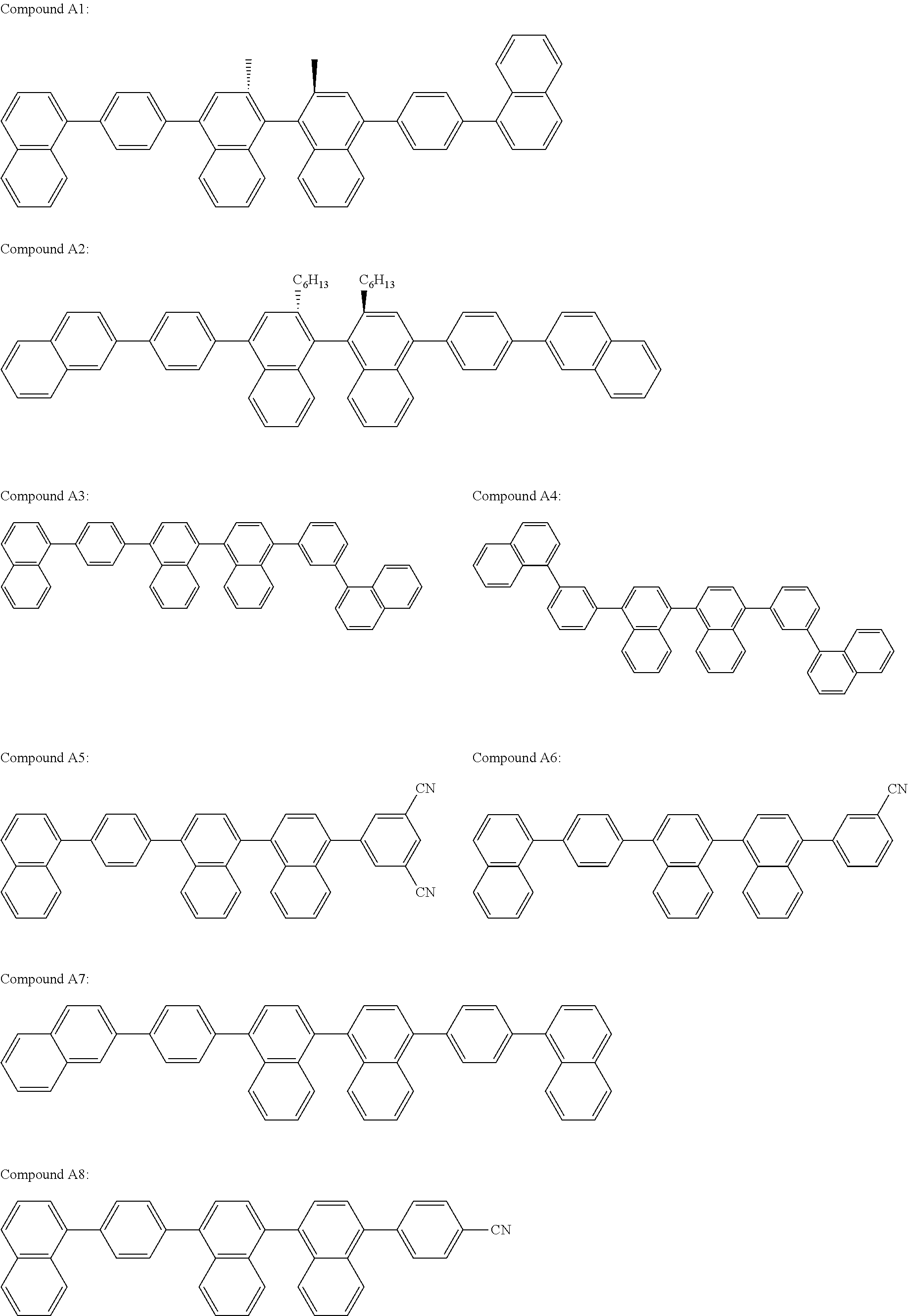

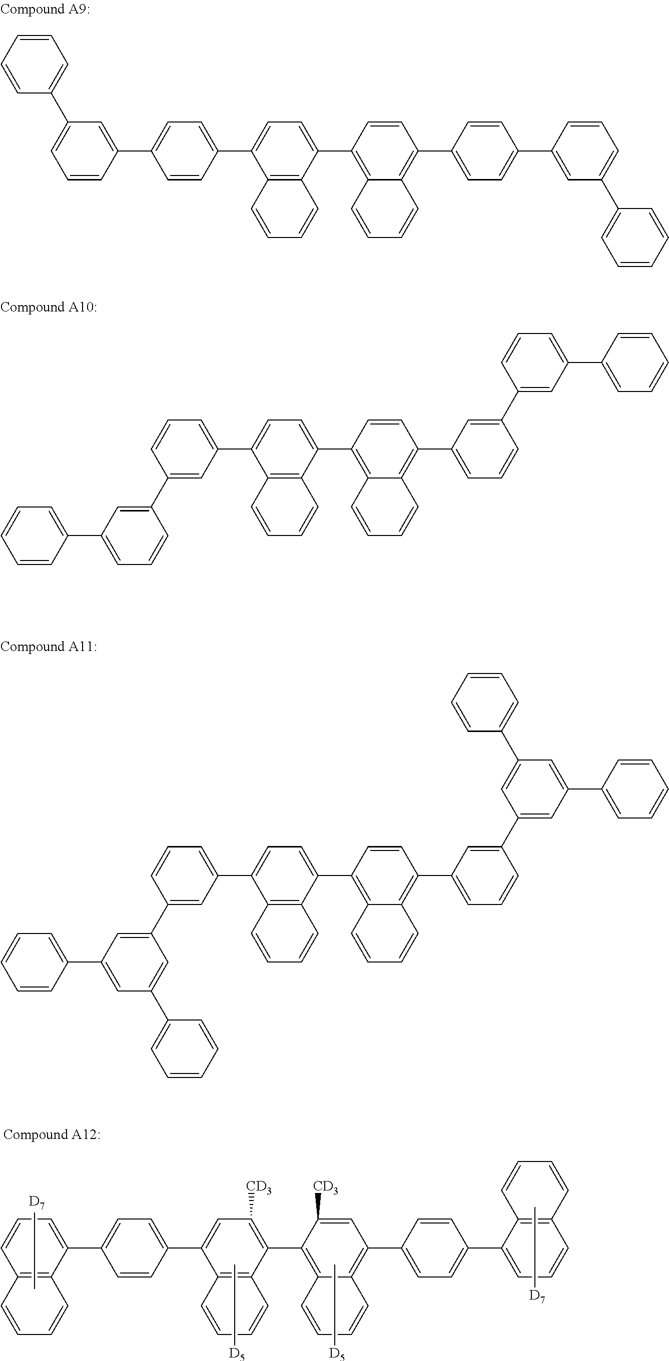

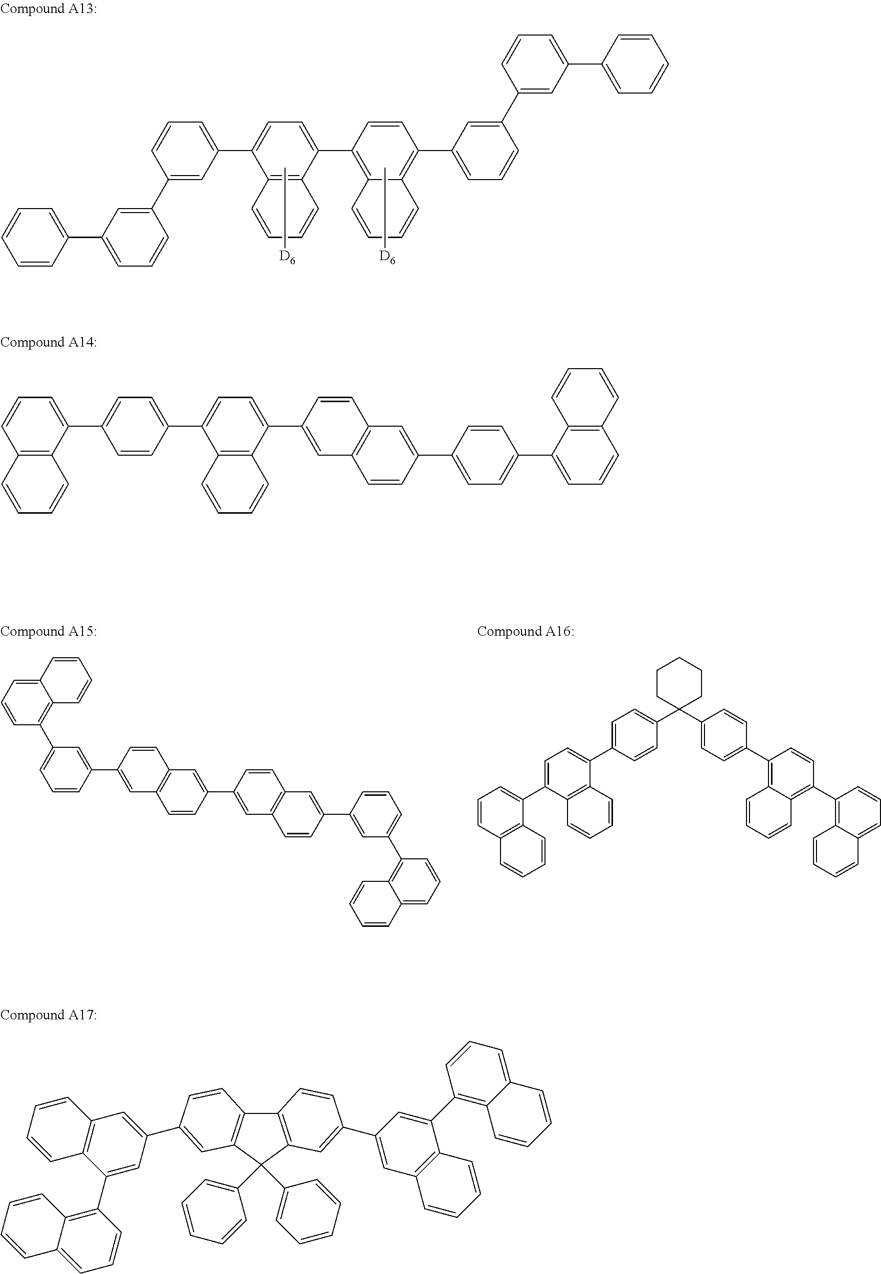

The new electroactive compounds can be prepared by known coupling and substitution reactions. The deuterated analog compounds can then be prepared in a similar manner using deuterated precursor materials or, more generally, by treating the non-deuterated compound with deuterated solvent, such as d6-benzene, in the presence of a Lewis acid H/D exchange catalyst, such as aluminum trichloride or ethyl aluminum chloride, or acids such as CF₃COOD, DCl, etc. Exemplary preparations are given in the Examples. The level of deuteration can be determined by NMR analysis and by mass spectrometry, such as Atmospheric Solids Analysis Probe Mass Spectrometry (ASAP-MS).

### 3. ELECTROACTIVE COMPOSITION

The electroactive composition described herein comprises: a host material and a dopant material, wherein the host material is a compound having one of Formulae I through VI

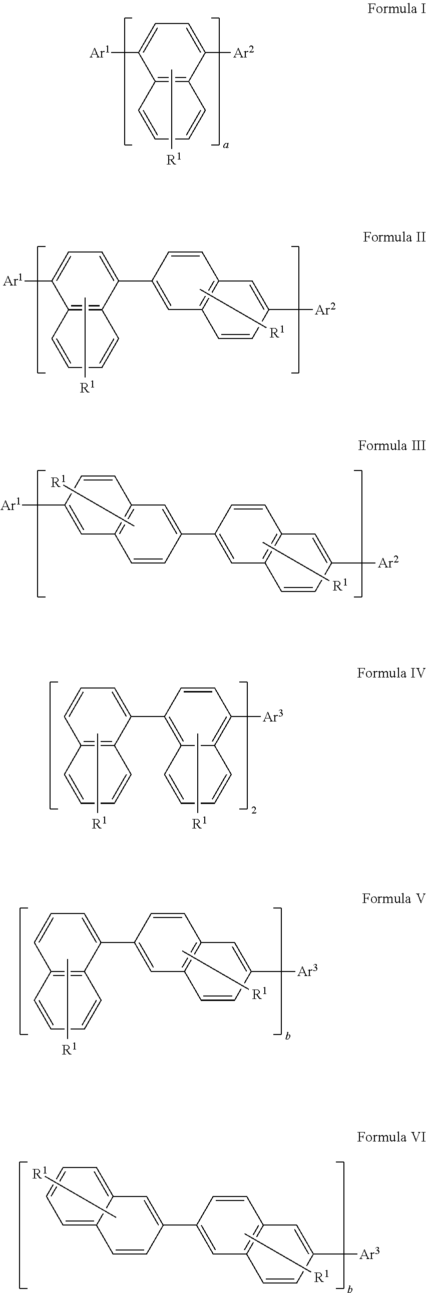

wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

In some embodiments, the electroactive composition consists essentially of a host material and a dopant material, wherein the host material is a compound having one of Formulae I-VI, described above.

In some embodiments, the host material having one of Formulae I-VI has a solubility in toluene of at least 0.6 wt %. In some embodiments, the solubility in toluene is at least 1 wt %.

In some embodiments, the host material has a Tg greater than 95°.

In some embodiments, the weight ratio of host material to the dopant is in the range of 5:1 to 25:1; in some embodiments, from 10:1 to 20:1.

In some embodiments, the electroactive composition further comprises a second host material. In some embodiments, the weight ratio of first host material to second host material is in the range of 99:1 to 1:99. In some embodiments, the ratio is in the range of 99:1 to 1.5:1; in some embodiments, 19:1 to 2:1; in some embodiments, 9:1 to 2.3:1. The first host material is different from the second host material. In some embodiments, the second host material is deuterated. In some embodiments, both the first and second host materials are deuterated. In some embodiments, the second host material is a phenanthroline, a quinoxaline, a phenylpyridine, a benzodifuran, a difuranobenzene, an indolocarbazole, a benzimidazole, a triazolopyridine, a diheteroarylphenyl, a metal quinolinate complexe, a substituted derivative thereof, a deuterated analog thereof, or a combination thereof.

In some embodiments, the electroactive composition comprises two or more electroluminescent dopant materials. In some embodiments, the composition comprises three dopants.

The compositions are useful as solution processable electroactive compositions for OLED devices. The resulting devices have high efficiency and long lifetimes. In some embodiments, the materials are useful in any printed electronics application including photovoltaics and TFTs.

The compounds described herein can be formed into films using liquid deposition techniques.

The dopant is an electroactive material which is capable of electroluminescence having an emission maximum between 380 and 750 nm. In some embodiments, the dopant emits red, green, or blue light. In some embodiments, the dopant is also deuterated.

In some embodiments, the dopant is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

Electroluminescent dopant materials include small molecule organic luminescent compounds, luminescent metal complexes, and combinations thereof. Examples of small molecule luminescent compounds include, but are not limited to, pyrene, perylene, rubrene, coumarin, derivatives thereof, and mixtures thereof. Examples of metal complexes include, but are not limited to, metal chelated oxinoid compounds, such as tris(8-hydroxyquinolato)aluminum (AlQ); cyclometalated iridium and platinum electroluminescent compounds, such as complexes of iridium with phenylpyridine, phenylquinoline, phenylisoquinoline or phenylpyrimidine ligands.

Examples of red light-emitting materials include, but are not limited to, cyclometalated complexes of Ir having phenylquinoline or phenylisoquinoline ligands, periflanthenes, fluoranthenes, and perylenes. Red light-emitting materials have been disclosed in, for example, U.S. Pat. No. 6,875,524, and published US application 2005-0158577.

Examples of green light-emitting materials include, but are not limited to, bis(diarylamino)anthracenes, and polyphenylenevinylene polymers. Green light-emitting materials have been disclosed in, for example, published PCT application WO 2007/021117.

Examples of blue light-emitting materials include, but are not limited to, diarylanthracenes, diaminochrysenes, diaminopyrenes, and polyfluorene polymers. Blue light-emitting materials have been disclosed in, for example, U.S. Pat. No. 6,875,524, and published US applications 2007-0292713 and 2007-0063638.

In some embodiments, the electroactive dopant is selected from the group consisting of a non-polymeric spirobifluorene compound, a fluoranthene compound, and deuterated analogs thereof.

In some embodiments, the electroactive dopant is a compound having aryl amine groups. In some embodiments, the electroactive dopant is selected from the formulae below:

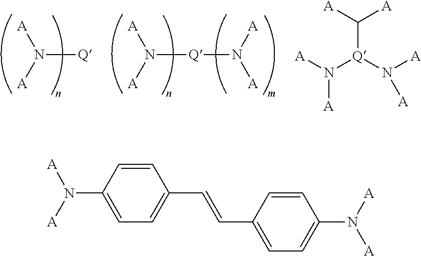

where:

A is the same or different at each occurrence and is an aromatic group having from 3-60 carbon atoms;

Q′ is a single bond or an aromatic group having from 3-60 carbon atoms;

n and m are independently an integer from 1-6.

In the above formula, the n and m may be limited by the number of available sites on the core Q′ group.

In some embodiments of the above formula, at least one of A and Q′ in each formula has at least three condensed rings. In some embodiments, m and n are equal to 1.

In some embodiments, Q′ is a styryl or styrylphenyl group.

In some embodiments, Q′ is an aromatic group having at least two condensed rings. In some embodiments, Q′ is selected from the group consisting of naphthalene, anthracene, benz[a]anthracene, dibenz[a,h]anthracene, fluoranthene, fluorene, spirofluorene, tetracene, chrysene, pyrene, tetracene, xanthene, perylene, coumarin, rhodamine, quinacridone, rubrene, substituted derivatives thereof, and deuterated analogs thereof.

In some embodiments, A is selected from the group consisting of phenyl, biphenyl, tolyl, naphthyl, naphthylphenyl, anthracenyl, and deuterated analogs thereof.

In some embodiments, the electroluminescent material has the structure

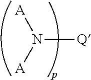

where A is an aromatic group, p is 1 or 2, and Q′ is

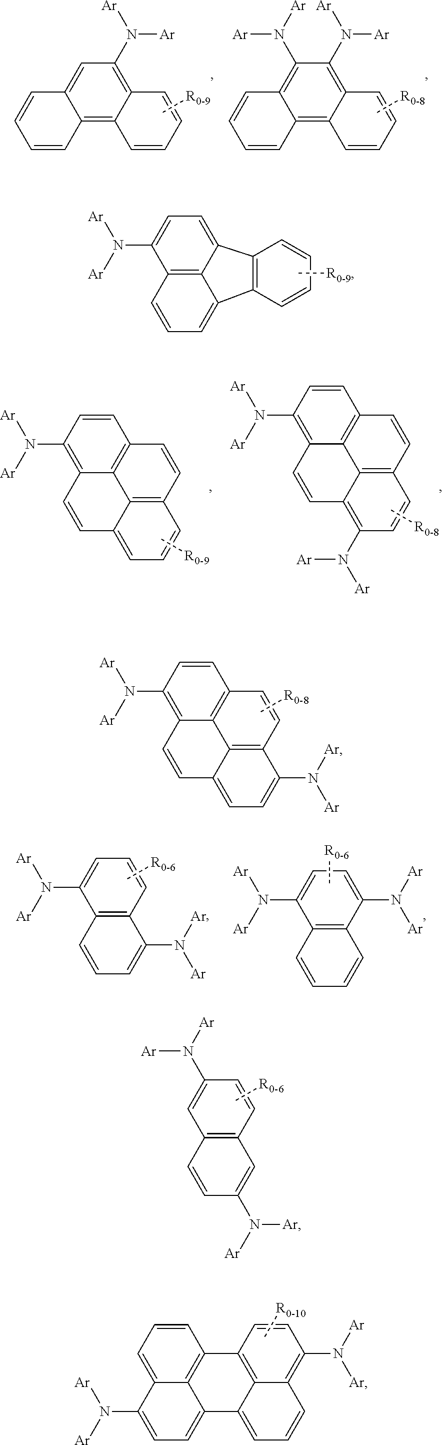

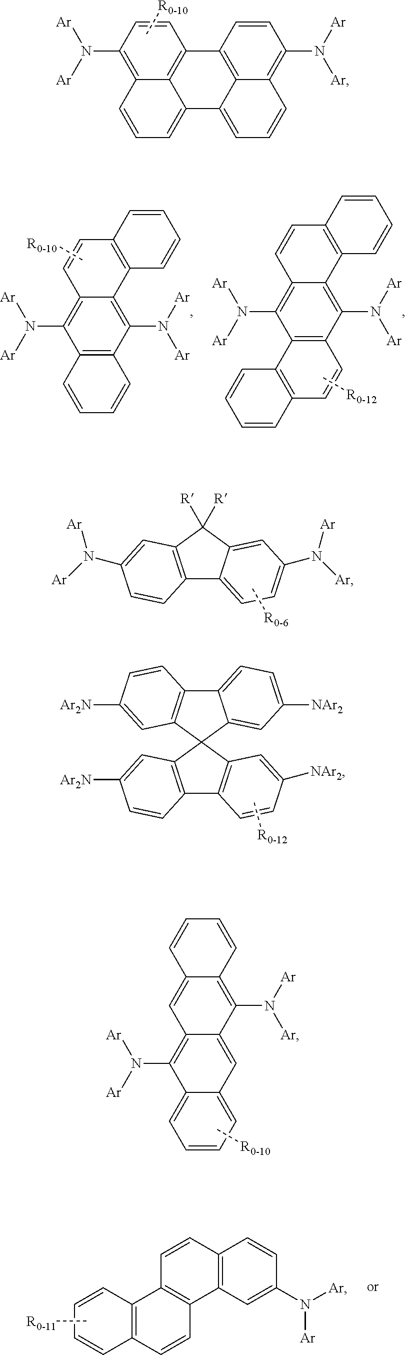

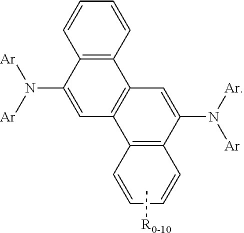

wherein:
R is the same or different at each occurrence and is D, alkyl, alkoxy or aryl, where adjacent R groups may be joined together to form a 5- or 6-membered aliphatic ring;
Ar is the same or different and is selected from the group consisting of aryl groups.

The dashed line in the formula is intended to indicate that the R group, when present, can be at any site on the core Q′ group.

In some embodiments, the electroactive dopant has the formula below:

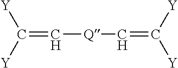

where:

Y is the same or different at each occurrence and is an aromatic group having 3-60 carbon atoms;

Q″ is an aromatic group, a divalent triphenylamine residue group, or a single bond.

In some embodiments, the electroactive dopant is an aryl acene. In some embodiments, the electroactive dopant is a non-symmetrical aryl acene.

In some embodiments, the electroactive dopant is a chrysene derivative. The term “chrysene” is intended to mean 1,2-benzophenanthrene. In some embodiments, the electroactive dopant is a chrysene having aryl substituents. In some embodiments, the electroactive dopant is a chrysene having arylamino substituents. In some embodiments, the electroactive dopant is a chrysene having two different arylamino substituents. In some embodiments, the chrysene derivative has a deep blue emission.

In some embodiments, separate electroactive compositions with different dopants are used to provide different colors. In some embodiments, the dopants are selected to have red, green, and blue emission. As used herein, red refers to light having a wavelength maximum in the range of 580-700 nm; green refers to light having a wavelength maximum in the range of 480-580 nm; and blue refers to light having a wavelength maximum in the range of 400-480 nm.

Examples of small molecule organic dopant materials include, but are not limited to, compounds D1 to D9 below.

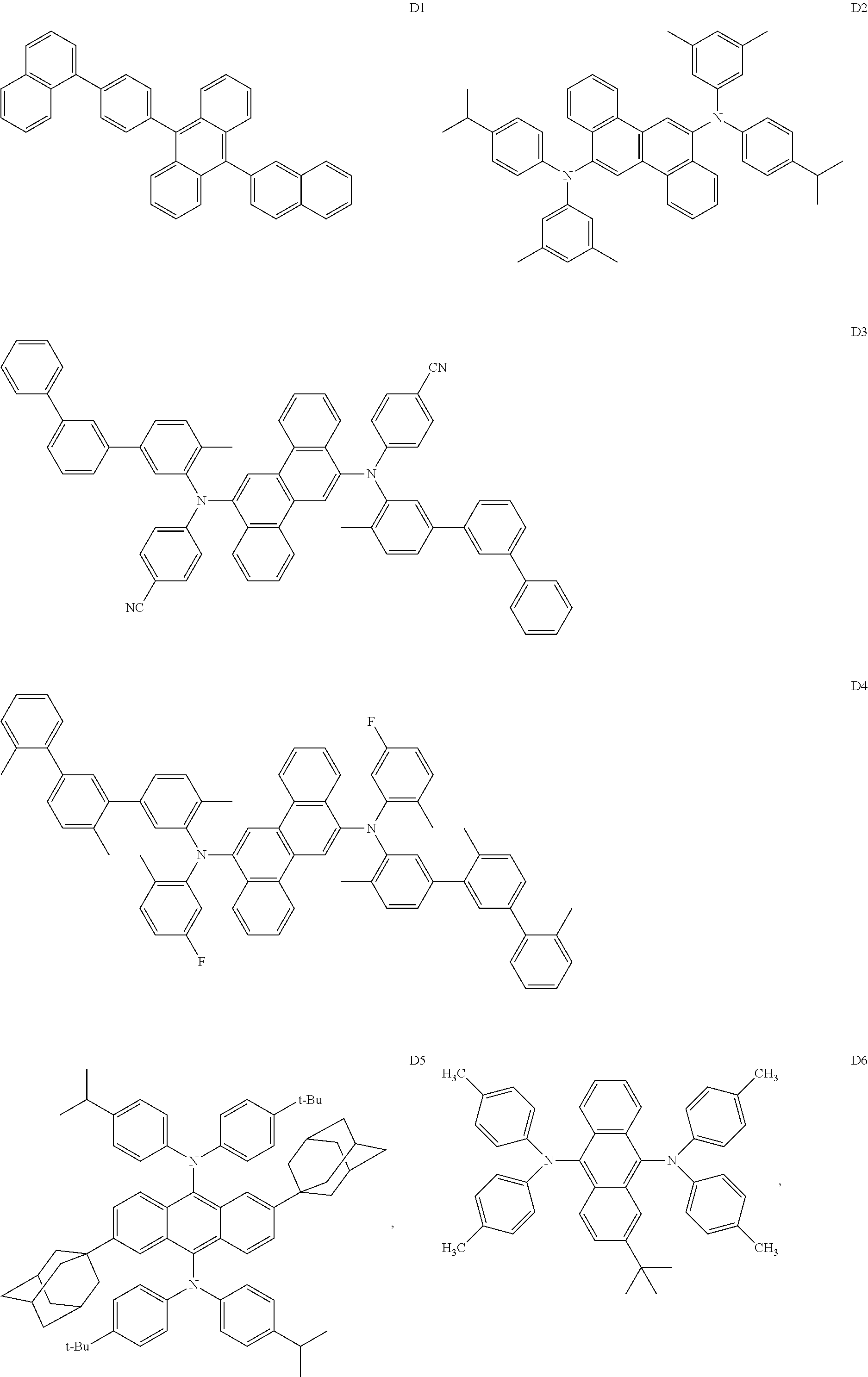

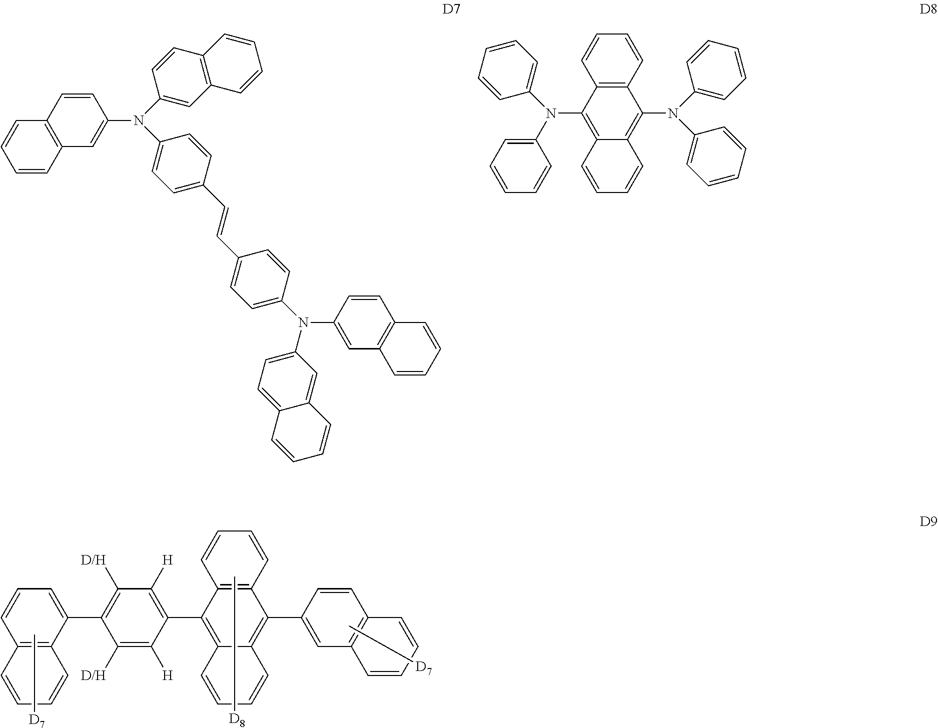

where “D/H” indicates approximately equal probability of H or D at this atomic position

### 4. ELECTRONIC DEVICE

Organic electronic devices that may benefit from having the electroactive composition described herein include, but are not limited to, (1) devices that convert electrical energy into radiation (e.g., a light-emitting diode, light emitting diode display, or diode laser), (2) devices that detect signals through electronics processes (e.g., photodetectors, photoconductive cells, photoresistors, photoswitches, phototransistors, phototubes, IR detectors, biosensors), (3) devices that convert radiation into electrical energy, (e.g., a photovoltaic device or solar cell), and (4) devices that include one or more electronic components that include one or more organic semi-conductor layers (e.g., a transistor or diode).

In some embodiments, an organic light-emitting device comprises:

an anode;

a hole transport layer;

a photoactive layer;

an electron transport layer, and

a cathode;

wherein the photoactive layer comprises the electroactive composition described above.

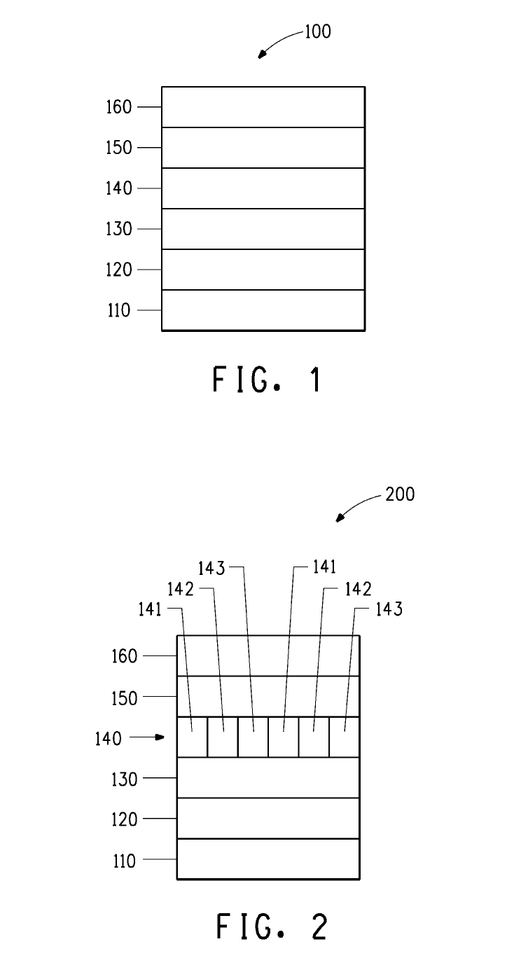

One illustration of an organic electronic device structure is shown in FIG. 1. The device 100 has a first electrical contact layer, an anode layer 110 and a second electrical contact layer, a cathode layer 160, and a photoactive layer 140 between them. Adjacent to the anode is a hole injection layer 120. Adjacent to the hole injection layer is a hole transport layer 130, comprising hole transport material. Adjacent to the cathode may be an electron transport layer 150, comprising an electron transport material. As an option, devices may use one or more additional hole injection or hole transport layers (not shown) next to the anode 110 and/or one or more additional electron injection or electron transport layers (not shown) next to the cathode 160.

Layers 120 through 150 are individually and collectively referred to as the active layers.

In some embodiments, the photoactive layer is pixellated, as shown in FIG. 2. In device 200, layer 140 is divided into pixel or subpixel units 141, 142, and 143 which are repeated over the layer. Each of the pixel or subpixel units represents a different color. In some embodiments, the subpixel units are for red, green, and blue. Although three subpixel units are shown in the figure, two or more than three may be used.

In one embodiment, the different layers have the following range of thicknesses: anode 110, 500-5000 Å, in one embodiment 1000-2000 Å; hole injection layer 120, 50-3000 Å, in one embodiment 200-1000 Å; hole transport layer 130, 50-2000 Å, in one embodiment 200-1000 Å; photoactive layer 140, 10-2000 Å, in one embodiment 100-1000 Å; layer 150, 50-2000 Å, in one embodiment 100-1000 Å; cathode 160, 200-10000 Å, in one embodiment 300-5000 Å. The location of the electron-hole recombination zone in the device, and thus the emission spectrum of the device, can be affected by the relative thickness of each layer. The desired ratio of layer thicknesses will depend on the exact nature of the materials used.

Depending upon the application of the device 100, the photoactive layer 140 can be a light-emitting layer that is activated by an applied voltage (such as in a light-emitting diode or light-emitting electrochemical cell), or a layer of material that responds to radiant energy and generates a signal with or without an applied bias voltage (such as in a photodetector). Examples of photodetectors include photoconductive cells, photoresistors, photoswitches, phototransistors, and phototubes, and photovoltaic cells, as these terms are described in Markus, John, Electronics and Nucleonics Dictionary, 470 and 476 (McGraw-Hill, Inc. 1966).

a. Photoactive Layer

The photoactive layer comprises the electroactive composition described above.

In some embodiments, the photoactive layer comprises a host material having one of Formulae I-VI and a dopant having deep blue emission. By “deep blue” is meant an emission wavelength of 420-475 nm. It has been found that the host compounds having one of Formulae I-VI can have a wide gap between HOMO and LUMO energy levels. This is advantageous when the dopant has deep blue emission and allows for emission of deep saturated blue color.

In some embodiments, the photoactive layer comprises a host material having one of Formulae I-VI and a chrysene dopant having deep blue emission. In some embodiments, the chrysene dopant is a bis(diarylamino)chrysene. In some embodiments, the photoactive layer consists essentially of a host material having one of Formulae I-VI and a chrysene dopant having deep blue emission. In some embodiments, the photoactive layer has an emission color with a y-coordinate less than 0.10, according to the C.I.E. chromaticity scale (Commission Internationale de L'Eclairage, 1931). In some embodiments, the y-coordinate is less than 0.7. The x-coordinate is in the range of 0.135-0.165.

The photoactive layer can be formed by liquid deposition from a liquid composition, as described below. In some embodiments, the photoactive layer is formed by vapor deposition.

In some embodiments, three different photoactive compositions are applied by liquid deposition to form red, green, and blue subpixels. In some embodiments, each of the colored subpixels is formed using new electroactive compositions as described herein. In some embodiments, the host materials are the same for all of the colors. In some embodiments, different host materials are used for the different colors.

b. Other Device Layers

The other layers in the device can be made of any materials that are known to be useful in such layers.

The anode 110, is an electrode that is particularly efficient for injecting positive charge carriers. It can be made of, for example, materials containing a metal, mixed metal, alloy, metal oxide or mixed-metal oxide, or it can be a conducting polymer, or mixtures thereof. Suitable metals include the Group 11 metals, the metals in Groups 4-6, and the Group 8-10 transition metals. If the anode is to be light-transmitting, mixed-metal oxides of Groups 12, 13 and 14 metals, such as indium-tin-oxide, are generally used. The anode 110 can also comprise an organic material such as polyaniline as described in “Flexible light-emitting diodes made from soluble conducting polymer,” Nature vol. 357, pp 477-479 (11 Jun. 1992). At least one of the anode and cathode is desirably at least partially transparent to allow the generated light to be observed.

The hole injection layer 120 comprises hole injection material and may have one or more functions in an organic electronic device, including but not limited to, planarization of the underlying layer, charge transport and/or charge injection properties, scavenging of impurities such as oxygen or metal ions, and other aspects to facilitate or to improve the performance of the organic electronic device. Hole injection materials may be polymers, oligomers, or small molecules. They may be vapour deposited or deposited from liquids which may be in the form of solutions, dispersions, suspensions, emulsions, colloidal mixtures, or other compositions.

The hole injection layer can be formed with polymeric materials, such as polyaniline (PANI) or polyethylenedioxythiophene (PEDOT), which are often doped with protonic acids. The protonic acids can be, for example, poly(styrenesulfonic acid), poly(2-acrylamido-2-methyl-1-propanesulfonic acid), and the like.

The hole injection layer can comprise charge transfer compounds, and the like, such as copper phthalocyanine and the tetrathiafulvalene-tetracyanoquinodimethane system (TTF-TCNQ).

In some embodiments, the hole injection layer comprises at least one electrically conductive polymer and at least one fluorinated acid polymer. Such materials have been described in, for example, published U.S. patent applications US 2004/0102577, US 2004/0127637, US 2005/0205860, and published PCT application WO 2009/018009.

Examples of hole transport materials for layer 130 have been summarized for example, in Kirk-Othmer Encyclopedia of Chemical Technology, Fourth Edition, Vol. 18, p. 837-860, 1996, by Y. Wang. Both hole transporting molecules and polymers can be used. Commonly used hole transporting molecules are: N,N′-diphenyl-N,N′-bis(3-methylphenyl)-[1,1′-biphenyl]-4,4′-diamine (TPD), 1,1-bis[(di-4-tolylamino) phenyl]cyclohexane (TAPC), N,N′-bis(4-methylphenyl)-N,N′-bis(4-ethylphenyl)-[1,1′-(3,3′-dimethyl)biphenyl]-4,4′-diamine (ETPD), tetrakis-(3-methylphenyl)-N,N,N′,N′-2,5-phenylenediamine (PDA), a-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl]pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), N,N′-bis(naphthalen-1-yl)-N,N′-bis-(phenyl)benzidine (α-NPB), and porphyrinic compounds, such as copper phthalocyanine. Commonly used hole transporting polymers are polyvinylcarbazole, (phenylmethyl)-polysilane, and polyaniline. It is also possible to obtain hole transporting polymers by doping hole transporting molecules such as those mentioned above into polymers such as polystyrene and polycarbonate. In some cases, triarylamine polymers are used, especially triarylamine-fluorene copolymers. In some cases, the polymers and copolymers are crosslinkable. In some embodiments, the hole transport layer further comprises a p-dopant. In some embodiments, the hole transport layer is doped with a p-dopant. Examples of p-dopants include, but are not limited to, tetrafluorotetracyanoquinodimethane (F4-TCNQ) and perylene-3,4,9,10-tetracarboxylic-3,4,9,10-dianhydride (PTCDA).

Examples of electron transport materials which can be used for layer 150 include, but are not limited to, metal chelated oxinoid compounds, including metal quinolate derivatives such as tris(8-hydroxyquinolato)aluminum (AlQ), bis(2-methyl-8-quinolinolato)(p-phenylphenolato) aluminum (BAlq), tetrakis-(8-hydroxyquinolato)hafnium (HfQ) and tetrakis-(8-hydroxyquinolato)zirconium (ZrQ); and azole compounds such as 2-(4-biphenylyl)-5-(4-t-butylphenyl)-1,3,4-oxadiazole (PBD), 3-(4-biphenylyl)-4-phenyl-5-(4-t-butylphenyl)-1,2,4-triazole (TAZ), and 1,3,5-tri(phenyl-2-benzimidazole)benzene (TPBI); quinoxaline derivatives such as 2,3-bis(4-fluorophenyl)quinoxaline; phenanthrolines such as 4,7-diphenyl-1,10-phenanthroline (DPA) and 2,9-dimethyl-4,7-diphenyl-1,10-phenanthroline (DDPA); and mixtures thereof. In some embodiments, the electron transport layer further comprises an n-dopant. N-dopant materials are well known. The n-dopants include, but are not limited to, Group 1 and 2 metals; Group 1 and 2 metal salts, such as LiF, CsF, and Cs₂CO₃; Group 1 and 2 metal organic compounds, such as Li quinolate; and molecular n-dopants, such as leuco dyes, metal complexes, such as W₂(hpp)₄ where hpp=1,3,4,6,7,8-hexahydro-2H-pyrimido-[1,2-a]-pyrimidine and cobaltocene, tetrathianaphthacene, bis(ethylenedithio)tetrathiafulvalene, heterocyclic radicals or diradicals, and the dimers, oligomers, polymers, dispiro compounds and polycycles of heterocyclic radical or diradicals.

The cathode 160, is an electrode that is particularly efficient for injecting electrons or negative charge carriers. The cathode can be any metal or nonmetal having a lower work function than the anode. Materials for the cathode can be selected from alkali metals of Group 1 (e.g., Li, Cs), the Group 2 (alkaline earth) metals, the Group 12 metals, including the rare earth elements and lanthanides, and the actinides. Materials such as aluminum, indium, calcium, barium, samarium and magnesium, as well as combinations, can be used. Li-containing organometallic compounds, LiF, and Li₂O can also be deposited between the organic layer and the cathode layer to lower the operating voltage.

It is known to have other layers in organic electronic devices. For example, there can be a layer (not shown) between the anode 110 and hole injection layer 120 to control the amount of positive charge injected and/or to provide band-gap matching of the layers, or to function as a protective layer. Layers that are known in the art can be used, such as copper phthalocyanine, silicon oxy-nitride, fluorocarbons, silanes, or an ultra-thin layer of a metal, such as Pt. Alternatively, some or all of anode layer 110, active layers 120, 130, 140, and 150, or cathode layer 160, can be surface-treated to increase charge carrier transport efficiency. The choice of materials for each of the component layers is preferably determined by balancing the positive and negative charges in the emitter layer to provide a device with high electroluminescence efficiency.

It is understood that each functional layer can be made up of more than one layer.

c. Device Fabrication

The device layers can be formed by any deposition technique, or combinations of techniques, including vapor deposition, liquid deposition, and thermal transfer. Substrates such as glass, plastics, and metals can be used. Conventional vapor deposition techniques can be used, such as thermal evaporation, chemical vapor deposition, and the like. The organic layers can be applied from solutions or dispersions in suitable solvents, using conventional coating or printing techniques, including but not limited to spin-coating, dip-coating, roll-to-roll techniques, ink-jet printing, continuous nozzle printing, screen-printing, gravure printing and the like.

In some embodiments, the process for making an organic light-emitting device, comprises:
providing a substrate having a patterned anode thereon;
forming a photoactive layer by depositing a first liquid composition comprising (a) a deuterated first host material, (b) an electroluminescent dopant material, and (c) a liquid medium; and
forming a cathode overall.

The term “liquid composition” is intended to include a liquid medium in which one or more materials are dissolved to form a solution, a liquid medium in which one or more materials are dispersed to form a dispersion, or a liquid medium in which one or more materials are suspended to form a suspension or an emulsion.

In some embodiments, the process further comprises:
forming a hole transport layer prior to forming the photoactive layer, wherein the hole transport layer is formed by depositing a second liquid composition comprising a hole transport material in a second liquid medium.

In some embodiments, the process further comprises:
forming an electron transport layer after forming the photoactive layer, wherein the electron transport layer is formed by depositing a third liquid composition comprising an electron transport material in a third liquid medium.

Any known liquid deposition technique or combination of techniques can be used, including continuous and discontinuous techniques. Examples of continuous liquid deposition techniques include, but are not limited to spin coating, gravure coating, curtain coating, dip coating, slot-die coating, spray coating, and continuous nozzle printing. Examples of discontinuous deposition techniques include, but are not limited to, ink jet printing, gravure printing, and screen printing. In some embodiments, the photoactive layer is formed in a pattern by a method selected from continuous nozzle coating and ink jet printing. Although the nozzle printing can be considered a continuous technique, a pattern can be formed by placing the nozzle over only the desired areas for layer formation. For example, patterns of continuous rows can be formed.

A suitable liquid medium for a particular composition to be deposited can be readily determined by one skilled in the art. For some applications, it is desirable that the compounds be dissolved in non-aqueous solvents. Such non-aqueous solvents can be relatively polar, such as C₁ to C₂₀ alcohols, ethers, and acid esters, or can be relatively non-polar such as C₁ to C₁₂ alkanes or aromatics such as toluene, xylenes, trifluorotoluene and the like. Another suitable liquid for use in making the liquid composition, either as a solution or dispersion as described herein, comprising the new compound, includes, but not limited to, a chlorinated hydrocarbon (such as methylene chloride, chloroform, chlorobenzene), an aromatic hydrocarbon (such as a substituted or non-substituted toluene or xylenes, including trifluorotoluene), a polar solvent (such as tetrahydrofuran (THF), N-methylpyrrolidone (NMP)), an ester (such as ethylacetate), an alcohol (such as isopropanol), a ketone (such as cyclopentatone), or any mixture thereof. Examples of mixtures of solvents for light-emitting materials have been described in, for example, published US application 2008-0067473.

In some embodiments, the weight ratio of total host material (first host together with second host, when present) to the dopant is in the range of 5:1 to 25:1.

After deposition, the material is dried to form a layer. Any conventional drying technique can be used, including heating, vacuum, and combinations thereof.

In some embodiments, the device is fabricated by liquid deposition of the hole injection layer, the hole transport layer, and the photoactive layer, and by vapor deposition of the anode, the electron transport layer, an electron injection layer and the cathode.

### EXAMPLES

The concepts described herein will be further described in the following examples, which do not limit the scope of the invention described in the claims.

### Synthesis Example 1

This example illustrates the preparation of compound A1.

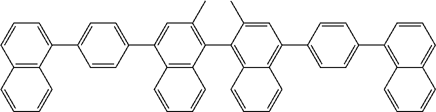

To a 500 mL round bottle flask were added 4,4′-dibromo-2,2′-dimethyl-1,1′-binaphthyl(4.40 g, 10 mmol), 4-(naphthalen-1-yl)phenylboronic acid (5.21 g, mmol), sodium carbonate (2 M, 30 mL, 60 mmol), toluene (120 mL) and Aliquat 336 (0.5 g). The mixture was system was stirred under nitrogen for 20 min. After which Tetrakis(triphenylphosphine) (462 mg, 0.4 mmol) was added and the mixture was stirred under nitrogen for another 15 min. The reaction was then heated in an oil bath at 95° C. for 18 hour. After cooling, the mixture was filtered through a Celite pad to remove the insoluble materials. The solution was washed with diluted HCl (10%, 80 mL), water (80 mL) and saturated brine (50 mL). The solvent was removed by rotary evaporation. The crude product was passed through a Silica gel column eluted with toluene. The product containing fractions were combined and the solvent was removed by rotary evaporation. Recrystallization from methylene chloride and acetonitrile gave the product as a white crystalline material. Yield, 2.6 g (38%). NMR spectra was consistent with the structure.

### Synthesis Example 2

This example illustrates the preparation of compound A4.

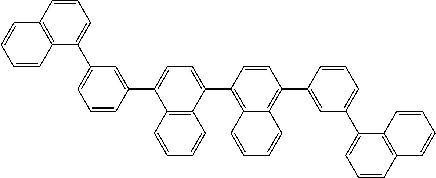

To a 500 mL round bottle flask were added 4,4′-dibromo-1,1′-binaphthyl (4.12 g, 10 mmol), 3-(naphthalen-1-yl)phenylboronic acid (5.21 g, mmol), sodium carbonate (2 M, 30 mL, 60 mmol), toluene (120 mL) and Aliquat 336 (0.5 g). The mixture was system was stirred under nitrogen for 20 min. After which Tetrakis(triphenylphospine) (462 mg, 0.4 mmol) was added and the mixture was stirred under nitrogen for another 15 min. The reaction was stirred and refluxed in an oil bath at 95° C. under nitrogen for 18 hour. After cooling to ambient temperature, some solid was seen formed and it was collected by filtration. The organic phase was separated, washed with water (60 mL), diluted HCl (10%, 60 mL) and saturated brine (60 mL) and dried with MgSO₄. The solution was filtered through a Silica gel plug and the solvent was removed by rotary evaporation. The solid collected earlier was triturated with hexane, filtered and combined with the residue from the liquid part. The material was re-dissolved in DCM/hexane and passed through a Silica gel column eluted with DCM/hexane. The product containing fractions were collected and the solvent was removed by rotary evaporation. The product was crystallized twice from toluene/EtOH to give the product as a white crystalline material. Yield, 2.60 g (39.52%). NMR spectra was consistent with the structure.

### Synthesis Example 3

This example illustrates the preparation of dopant D3.

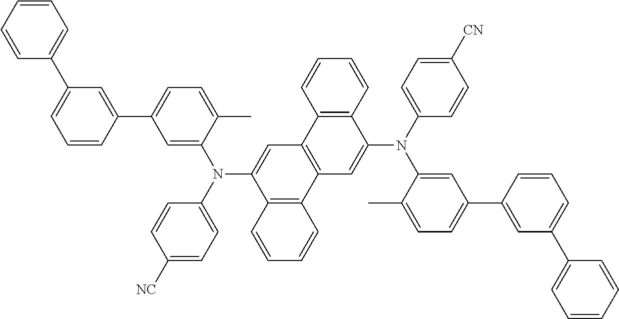

To a 50 mL round bottle flask were added palladium acetate (49 mg, 0.22 mmol), S-Phos (267 mg, 0.65 mmol), water (150 mg, 0.87 mmol) and dioxane (5 mL). The mixture was heated to 8 C. and stirred under nitrogen for 15 min. During the time the solution turned from orange red to dark red.

To a separate 500 mL round bottle flask were added 3-chloro-3′-phenyl-4-methylbiphenyl (6.44 g, 21.71 mmol), 4-aminobenzonitrile (3.14 g, 26.06 mmol), and dioxane (200 mL). With stirring under nitrogen, above catalyst solution was added followed by the sodium t-butoxide (2.71 g, 28.23 mmol). The reaction was then stirred at 95 C under nitrogen for 18 hour. After cooling to ambient temperature, the mixture was passed through a Celite plug, eluted with chloroform and the solvent was removed by rotary evaporation. The residue was dissolved in chloroform (10 mL) and hexanes (20 mL) and separated on a short silica gel column eluted with chloroform/hexanes (1/2). The product containing fractions were collected and the solvent was removed by rotary evaporation to give 7.28 g of 4-(3′-phenyl-4-methylbiphenyl-3-ylamino)benzonitrile in 99% purity by HPLC analysis. NMR spectra were consistent with the structure. To a 250 mL round bottle flask were added 4-(3′-phenyl-4-methylbiphenyl-3-ylamino)benzonitrile (3.60 g, 9.9 mmol), 6,12-dibromochrysene (1.85 g, 4.71 mmol, Pd₂(dba)₃ (86 mg, 0.094 mmol), tri-t-butylphosphine (38.10 mg, 0.19 mmol) and toluene (47 mL). The mixture was stirred under nitrogen for 1 min followed by addition of sodium t-butoxide (1.00 g, 10.36 mmol). The reaction was stirred and heated at 80° C. under nitrogen for 18 hour. After cooing to ambient temperature, the mixture was passed through a layer of Celite and a layer of Silica gel eluted with chloroform. The solvent was removed by rotary evaporation, and the residue was separated by chromatography on Silica gel column eluted with chloroform/hexane gradients. The product containing fractions were collected and the solvent was removed by rotary evaporation. The residue was recrystallized from toluene/EtOH to give the product as a white crystalline material. Yield, 0.25 g in >99% purity and 1.59 g in >97% purity. NMR spectra were consistent with the structure.

### Synthesis Example 4

This example illustrates the preparation of dopant D4.

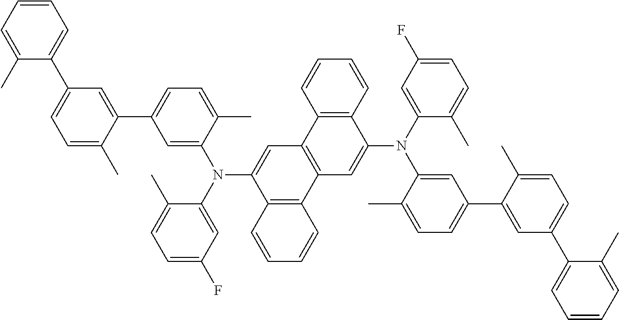

To a 50 mL round bottle flask were added palladium acetate (28 mg, 0.12 mmol), S-Phos (154 mg, 0.38 mmol), water (90 mg, 0.50 mmol) and dioxane (2 mL). The mixture was heated to 8 C. and stirred under nitrogen for 15 min. During the time the solution turned from pale orange to dark red.

To a separate 500 mL round bottle flask were added 3′-chloro-5-O— tolyl-2,4′-dimethylbiphenyl (4.00 g, 96%, 12.52 mmol), 5-fluoro-2-methylaniline (1.90 g, 15.02 mmol), and dioxane (200 mL). With stirring under nitrogen, above catalyst solution was added followed by the sodium t-butoxide (1.56 g, 16.27 mmol). The reaction was then stirred at 90 C under nitrogen for 18 hour. After cooling to ambient temperature, the mixture was passed through a Celite plug, eluted with chloroform and the solvent was removed by rotary evaporation. The residue was dissolved in dichloromethane/hexanes (1/1, 20 mL) and separated on a short silica gel column eluted with hexane first and then with chloroform/hexanes (1/1). The product containing fractions were collected and the solvent was removed by rotary evaporation to give 4.95 g of 5′-o-tolyl-N-(5-fluoro-2-methylphenyl)-2′,4-dimethylbiphenyl-3-amine in 99% purity by HPLC analysis. NMR spectra were consistent with the structure. To a 250 mL round bottle flask were added of 5′-o-tolyl-N-(5-fluoro-2-methylphenyl)-2′,4-dimethylbiphenyl-3-amine (2.47 g, 6.209 mmol), 6,12-dibromochrysene (1.11 g, 2.80 mmol, Pd₂(dba)₃ (56 mg, 0.062 mmol), tri-t-butylphosphine (23 mg, 0.11 mmol) and toluene (62 mL). The mixture was stirred under nitrogen for 1 min followed by addition of sodium t-butoxide (0.60 g, 6.20 mmol). The reaction was stirred and heated at 80° C. under nitrogen for 18 hour. After cooing to ambient temperature, the mixture was passed through a layer of Celite and a layer of Silica gel eluted with chloroform. The solvent was removed by rotary evaporation, and the residue was separated by chromatography on Silica gel column eluted with chloroform/hexane gradients. The product containing fractions were collected and the solvent was removed by rotary evaporation. The residue was recrystallized from chloroform/EtOH to give the product as a white crystalline material. Yield, 0.26 g in >99% purity. NMR spectra were consistent with the structure.

### Synthesis Example 5

This example illustrates the preparation of dopant D9.

### (a) Synthesis of Intermediate 1

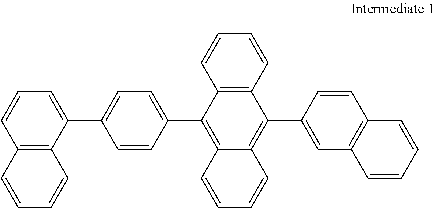

To a 250 mL flask in glove box were added (2.00 g, 5.23 mmol), 4,4,5,5-tetramethyl-2-(4-(naphthalen-4-yl)phenyl)-1,3,2-dioxaborolane (1.90 g, 5.74 mmol), tris(dibenzylideneacetone)dipalladium(0) (0.24 g, 0.26 mmol), and toluene (50 mL). The reaction flask was removed from the dry box and fitted with a condenser and nitrogen inlet. Degassed aqueous sodium carbonate (2 M, 20 mL) was added via syringe. The reaction was stirred and heated to 90° C. overnight. The reaction was monitored by HPLC. After cooling to room temperature, the organic layer was separated. The aqueous layer was washed twice with DCM and the combined organic layers were concentrated by rotary evaporation to afford a grey powder. Purification by filtration over neutral alumina, hexanes precipitation, and column chromatography over silica gel afforded 2.28 g of a white powder (86%).

The product was further purified as described in published U.S. patent application 2008-0138655, to achieve an HPLC purity of at least 99.9% and an impurity absorbance no greater than 0.01.

### (b) Synthesis of Dopant D9

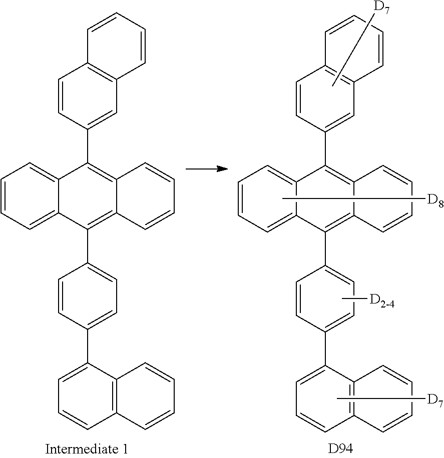

Under an atmosphere of nitrogen, AlCl₃ (0.48 g, 3.6 mmol) was added to a perdeuterobenzene or benzene-D6 (C₆D₆) (100 mL) solution of Intermediate 1 (5 g, 9.87 mmol). The resulting mixture was stirred at room temperature for six hours after which D₂O (50 mL) was added. The layers were separated followed by washing the water layer with CH₂Cl₂ (2×30 mL). The combined organic layers were dried over magnesium sulfate and the volatiles were removed by rotary evaporation. The crude product was purified via column chromatography. The deuterated product D9 (x+y+n+m=21-23) was obtained (4.5 g) as a white powder.

The product was further purified as described in published U.S. patent application 2008-0138655, to achieve an HPLC purity of at least 99.9% and an impurity absorbance no greater than 0.01. The material was determined to have the same level of purity as Intermediate 1, from above. The structure was confirmed by ¹H NMR, ¹³C NMR, ²D NMR and ¹H-¹³C HSQC (Heteronuclear Single Quantum Coherence).

### Device Examples 1-3 and Comparative Examples A-B

These examples demonstrate the fabrication and performance of OLED devices. For the comparative examples, D9 was used as the host. The non-deuterated analogs of such materials have been previously disclosed as blue host materials in, for example, published U.S. patent application no. US 2007-0088185.

The devices had the following structure on a glass substrate:
anode=Indium Tin Oxide (ITO), 50 nm
hole injection layer=HIJ-1 (50 nm), which is an electrically conductive polymer doped with a polymeric fluorinated sulfonic acid. Such materials have been described in, for example, published U.S. patent applications US 2004/0102577, US 2004/0127637, US 2005/0205860, and published PCT application WO 2009/018009.
hole transport layer=HT-1 (20 nm), which is a triarylamine-containing copolymer. Such materials have been described in, for example, published PCT application WO 2009/067419.
photoactive layer is shown in Table 1 (20 nm).
electron transport layer=ET-1 (10 nm), which is a phenanthroline derivative
electron injection layer/cathode=CsF/Al (0.7/100 nm)

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen.

Immediately before device fabrication the cleaned, patterned ITO substrates were treated with UV ozone for 10 minutes. Immediately after cooling, an aqueous dispersion of HIJ-1 was spin-coated over the ITO surface and heated to remove solvent. After cooling, the substrates were then spin-coated with a solution of a hole transport material, and then heated to remove solvent. After cooling the substrates were spin-coated with solution of the photoactive layer materials in methyl benzoate and heated to remove solvent. The substrates were masked and placed in a vacuum chamber. The electron transport layer was deposited by thermal evaporation, followed by a layer of CsF. Masks were then changed in vacuo and a layer of Al was deposited by thermal evaporation. The chamber was vented, and the devices were encapsulated using a glass lid, desiccant, and UV curable epoxy.

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The results are given in Table 2.

TABLE 1
Photoactive Layer

| | | | Host:Dopant |
|---|---|---|---|
| Device Example | Host | Dopant | weight ratio |
| 1 | A1 | D3 | 77:23 |
| 2 | A1 | D4 | 77:23 |
| 3 | A4 | D9 | 90:10 |
| Comp. A | D9 | D3 | 86:14 |
| Comp. B | D9 | D4 | 86:14 |

TABLE 2
Device Results

| Device | | | E.Q.E | C.E. | C.I.E. |
|---|---|---|---|---|---|
| Example | Host | Dopant | (%) | (cd/A) | x, y |
| 1 | A1 | D3 | 2.9 | 1.5 | 0.151, 0.052 |
| Comp. A | D9 | D3 | 3.6 | 3.4 | 0.147, 0.110 |
| 2 | A1 | D4 | 2.9 | 1.5 | 0.151, 0.053 |
| Comp. B | D9 | D4 | 3.8 | 3.7 | 0.146, 0.111 |
| 3 | A4 | D9 | 2.0 | 1.0 | 0.152, 0.055 |

All data @ 1000 nits; E.Q.E is the external quantum efficiency C.E. = current efficiency; CIEx and CIEy are the x and y color coordinates according to the C.I.E. chromaticity scale (Commission Internationale de L'Eclairage, 1931).

As can be seen from the table, a deeper blue color with a lower CIEy value is achieved in the devices having the host materials described herein.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. An electroactive compound having one of Formulae I through VI 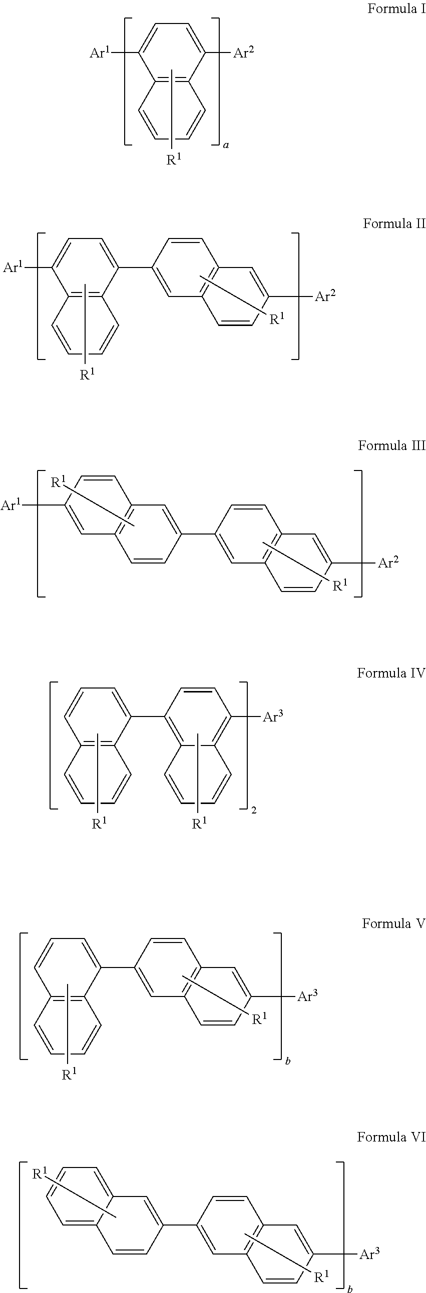
wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

2. The compound of claim 1, wherein the compound is at least 10% deuterated.

3. The compound of claim having one of Formulae I through III, wherein Ar¹ and Ar² are the same or different and have Formula a: 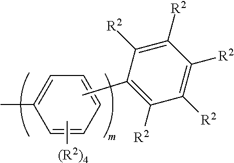
where:
R² is the same or different at each occurrence and is H, D, alkyl, alkoxy, siloxane or silyl, or adjacent R² groups may be joined together to form an aromatic ring; and
m is the same or different at each occurrence and is an integer from 1 to 6.

4. The compound of claim 1 having one of Formulae I through III, wherein Ar¹ and Ar² are the same or different and are phenyl, biphenyl, naphthylphenyl, naphthylbiphenyl, terphenyl, or quaterphenyl.

5. The compound of claim 1 having one of Formulae IV through VI, wherein Ar³ has Formula b: 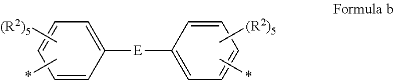
where:
R² is the same or different at each occurrence and is H, D, alkyl, alkoxy, siloxane or silyl, or adjacent R² groups may be joined together to form an aromatic ring;
E is a single bond, C(R³)₂, O, Si(R³)₂, or Ge(R³)₂;
R³ is alkyl or aryl, or two R³ groups can join together to form a non-aromatic ring; and
the asterisk indicates the point of attachment to the remainder of the compound.

6. The compound of claim 1, wherein there is at least one substituent on at least one aryl ring, and the substituent is D, alkyl, alkoxy, siloxane or silyl.

7. The compound of claim 1, wherein is the compound is one of Compound A1 through Compound A17.

8. An electroactive composition comprising a host material and a dopant material, wherein the host material is a compound having one of Formulae I through VI 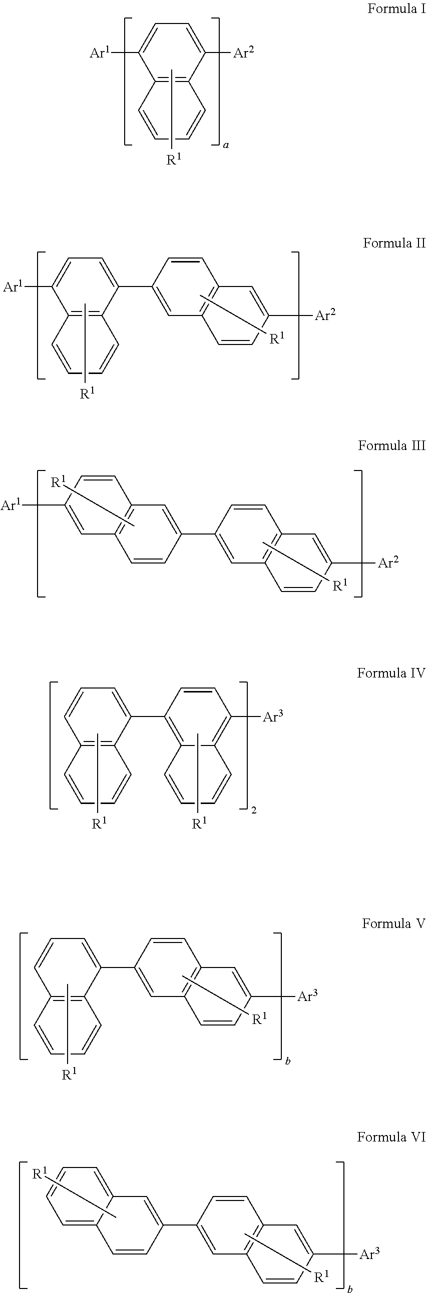
wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

9. The composition of claim 8, wherein the dopant has deep blue emission.

10. The composition of claim 9, wherein the dopant is a chrysene derivative.

11. An organic light-emitting device comprising two electrical contact layers with an organic photoactive layer therebetween, wherein the photoactive layer comprises a host material and a dopant material, wherein the host material is a compound having one of Formulae I through VI 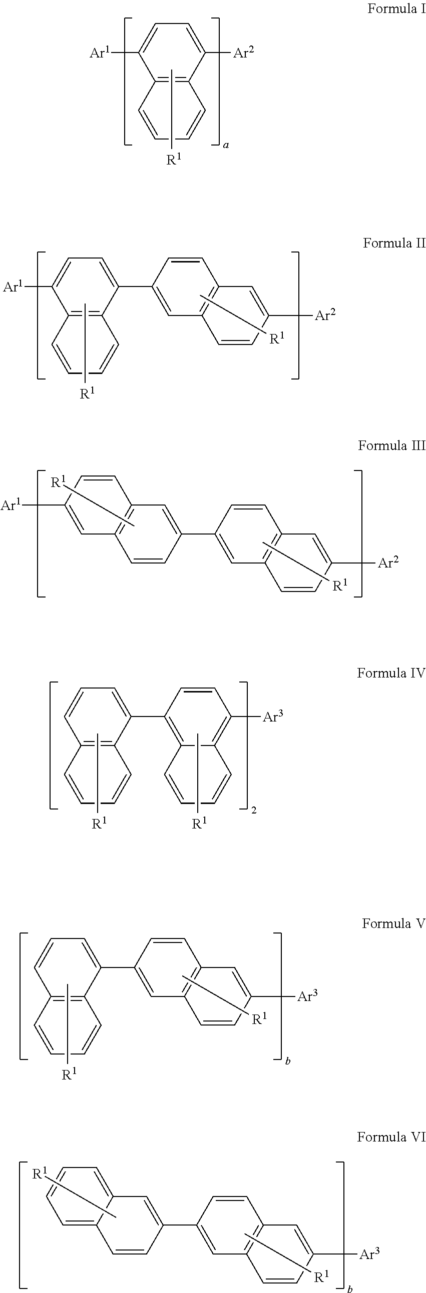
wherein:
R¹ represents 0-z substituents on the aromatic group, where z is the maximum number of substituent positions available, and R¹ is the same or different at each occurrence and is D, alkyl, aryl, alkoxy, aryloxy, oxyalkyl, alkenyl, silyl, or siloxane;
Ar¹, Ar², and Ar³ are the same or different at each occurrence and are aryl groups;
a is an integer from 2-6; and
b is an integer from 1-3.

12. The device of claim 11, wherein the dopant has deep blue emission.

13. The device of claim 12, wherein the emission color has a y-coordinate less than 0.10, according to the C.I.E. chromaticity scale.
